(19) United States
(12) Patent Application Publication
(10) Pub. No.: US 2002/0032934 A1
(43) Pub. Date: Mar. 21, 2002

(54) Composition for oxidation dyeing of keratin fibers, comprising at least one oxidation base chosen from 5-methylpyrazolo(1,5-a)pyrimidine-3,7-diamine and addition salts thereof; dyeing processes using them

(76) Inventors: Sylvain Kravtchenko, Asnieres (FR); Alain Lagrange, Coupvray (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/860,585

(22) Filed: May 21, 2001

(30) Foreign Application Priority Data
May 19, 2000 (FR) 00 07120

(51) Int. Cl. A61K 7/13

(52) U.S. Cl. 8/405; 8/406; 8/416

(57) ABSTRACT

The invention relates to a composition for the oxidation dyeing of keratin fibers, and for example human keratin fibers chosen from hair, containing at least on oxidation base chosen from 5-methylpyrazolo(1,5-a)pyrimidine-3,7-diamine and addition salts thereof; the composition containing no coupler chosen from the N-(2-hydroxybenzene)carbamate type and the N-(2-hydroxybenzene)urea type, and to oxidation dyeing processes using it.

The invention relates to a composition for the oxidation dyeing of keratin fibers, for example human keratin fibers chosen from hair, comprising at least one oxidation base chosen from 5-methylpyrazolo(1,5-a)pyrimidine-3,7-diamine and addition salts thereof; the composition containing no coupler of the N-(2-hydroxybenzene)carbamate type or of the N-(2-hydroxybenzene)urea type, and to oxidation dyeing processes using it.

It is a known practice to dye keratin fibers, for example human hair, with dye compositions containing oxidation dye precursors, for example ortho-phenylenediamines, para-phenylenediamines, ortho-aminophenols, para-aminophenols, and heterocyclic compounds chosen from diaminopyrazole derivatives, generally known as oxidation bases. The oxidation dye precursors, or oxidation bases, are colourless and weakly coloured compounds which, when combined with oxidizing products, can give rise to coloured compounds and dyes by a process of oxidative condensation.

It is also known that the shades obtained with these oxidation bases can be varied by combining them with couplers and coloration modifiers, the latter being chosen from, for example, aromatic meta-diamines, meta-aminophenols, meta-diphenols and certain heterocyclic compounds.

The variety of molecules used as oxidation bases and couplers can allow a wide range of colours to be obtained.

The so-called “permanent” coloration obtained by means of these oxidation dyes should have no toxicological drawbacks, should allow shades to be obtained in the desired intensity, and/or should have good staying power with respect to external agents (light, bad weather, washing, permanent-waving, perspiration or rubbing).

The dyes should also allow grey hair to be covered, and should be as unselective as possible, i.e., they should allow only the smallest possible differences in coloration along the same keratin fiber, which may indeed be differently sensitized (i.e., damaged) between its tip and its root. The dyes should also have good chemical stability in the formulations and should have a good toxicological profile.

It has already been proposed, for example in patent application DE 4 029 324, the disclosure of which is hereby incorporated by reference, to use certain pyrazolo(1,5-a)pyrimidine derivatives, which may be substituted with (C₁-C₄) alkyl radicals in position 4, 5 and/or 6, as couplers for the oxidation dyeing of keratin fibers.

It has also been proposed in patent application DE 4 133 957, to use certain pyrazolo(1,5-a)pyrimidine derivatives belonging to the tetrahydropyrazolo(1,5-a)pyrimidine family as oxidation dye precursors for the oxidation dyeing of keratin fibers.

Patent application EP-A-0 847 271, recommends using certain pyrazolo-(1,5-a)pyrimidine derivatives chosen from pyrazolo(1,5-a)pyrimidine-3,7-diamine as oxidation bases for the oxidation dyeing of keratin fibers. These derivatives also are reported to have a good capacity to dye keratin fibers in a resistant and intense manner, and also a good toxicological profile. However, pyrazolo(1,5-a)pyrimidine derivatives generally have problems of solubility in dye supports, which considerably limits their level of incorporation into the formulations and the possibility of obtaining colorations with the desired intensity.

The inventors have now discovered, entirely surprisingly and unexpectedly, that it is possible to obtain dyes with at least one of the following properties: that are capable of giving intense, particularly chromatic and bright colorations, that are relatively unselective, and that have excellent properties of resistance to the various attacking factors to which keratin fibers may be subjected. The dyes can be obtained by using, for example, at least one oxidation base chosen from pyrazolo(1,5-a)pyrimidine whose solubility is substantially greater than that of the pyrazolo(1,5-a)pyrimidines mentioned above.

The at least one oxidation base is chosen from 5-methylpyrazolo(1,5-a)- pyrimidine-3,7-diamine of formula (I):

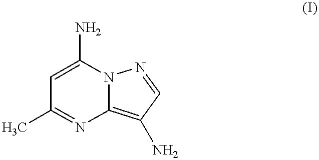

acid addition salts thereof, and base addition salts thereof.

A first subject of the invention is thus a composition for the oxidation dyeing of keratin fibers, for example, human keratin fibers chosen from hair, comprising, in, for example, a medium which is suitable for dyeing, at least one oxidation base chosen from 5-methylpyrazolo(1,5-a)pyrimidine-3,7-diamine of formula (I):

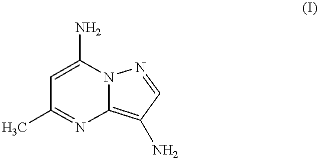

acid addition salts thereof, and base addition salts thereof; the composition containing no coupler chosen from the N-(2-hydroxybenzene)carbamate type and the N-(2-hydroxy-benzene)urea type.

As mentioned above, the colorations obtained with the oxidation dye composition in accordance with the invention may be intense, particularly bright, and chromatic. They make it possible, for example, to obtain red shades that may be free of and contain very little blue or yellow. Furthermore, they may also have excellent properties of resistance with respect to the action of various external agents (light, bad weather, washing, permanent-waving, perspiration and rubbing).

A subject of the invention is also a process for the oxidation dyeing of keratin fibers using this dye composition.

The at least one oxidation base may be present in the dye composition in an amount ranging, for example, from 0.001% to 10% by weight approximately relative to the total weight of the dye composition, such as ranging, for example, from 0.05% to 6% by weight approximately relative to the total weight of the dye composition.

The dye composition in accordance with the invention may also contain, in addition to the dyes defined above, at least one additional oxidation base which may be chosen from oxidation bases conventionally used in oxidation dyeing and among which mention may be made, for example, of para-phenylenediamines, bis(phenyl)alkylenediamines, para-aminophenols, ortho-aminophenols, and heterocyclic bases other than the 5-methylpyrazolo(1,5-a)pyrimidine-3,7-diamine used in accordance with the invention.

Among the para-phenylenediamines which may be mentioned, more particularly, by way of example, are para-phenylenediamine, para-tolylenediamine, 2,6-dimethyl-para-phenylenediamine, 2-β-hydroxyethyl-para-phenylenediamine, 2-n-propyl-para-phenylenediamine, 2-isopropyl-para-phenylenediamine, N-(β-hydroxypropyl)-para-phenylenediamine, N,N-bis(β-hydroxyethyl)-para-phenylenediamine, 4-amino-N-(β-methoxyethyl)aniline, and the para-phenylenediamines described in French patent application FR 2 630 438, the disclosure of which relating to para-phenylenediamines, is hereby incorporated by reference, and the addition salts thereof.

Among the bis(phenyl)alkylenediamines which may be mentioned, more particularly, by way of example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-amino-phenyl)tetramethylenediamine, N,N′-bis(4-methylaminophenyl)tetramethylenediamine, and N,N′-bis(ethyl)-N,N′-bis(4′-amino-3′-methylphenyl)ethylenediamine, and the addition salts thereof.

Among the para-aminophenols which may be mentioned, more particularly, by way of example, are para-aminophenol, 4-amino-3-methylphenol, 4-amino-3-fluoro-phenyl, 4-amino-3-hydroxymethylphenol, 4-amino-2-methylphenol, 4-amino-2-hydroxymethylphenol, 4-amino-2-methoxymethyl phenol, 4-am ino-2-aminomethylphenol, and 4-amino-2-(β-hydroxyethylaminomethyl)phenol, and the addition salts thereof.

Among the ortho-aminophenols which may be mentioned, more particularly, by way of example, are 2-aminophenol, 2-amino-5-methylphenol, 2-amino-6-methylphenol, and 5-acetamido-2-aminophenol, and the addition salts thereof.

Among the heterocyclic bases which may be mentioned, more particularly, by way of example, are pyridine derivatives, pyrimidine derivatives, pyrazole derivatives other than the pyrazolo(1,5-a)pyrimidine derivatives of formula (I) used in accordance with the invention, for example, chosen from 4,5-diaminopyrazoles, and the addition salts thereof.

When it is used, the at least one additional oxidation base may be present in the dye composition in an amount ranging, for example, from 0.0005% to 12% by weight approximately relative to the total weight of the dye composition, and as a further example, ranging from 0.005% to 6% by weight approximately relative to the total weight of the dye composition.

The oxidation dye composition in accordance with the invention, may also contain at least one coupler and/or at least one direct dye, for example, to at least modify the shades and to enrich them with glints.

The at least one coupler which may be used in the oxidation dye composition, in accordance with the invention, may be chosen from couplers used conventionally in oxidation dyeing and among which mention may be made, for example, of meta-phenylenediamines, meta-aminophenols, metadiphenols, mononapthalene derivatives, polyhydroxylated naphthalene derivatives, and heterocyclic couplers, chosen from, for example, indole derivatives, pyridine derivatives, and the acid addition salts thereof.

The at least one coupler is chosen more particularly from 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxy-ethylamino)-1-methoxybenzene, 1,3-diaminobenzene, 1,3-bis(2,4-diaminophenoxy)-propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine and 6-hydroxy-benzomorpholine, and the addition salts thereof.

When it is present, the at least one coupler may be present in the dye composition in an amount ranging for example, from 0.0001% to 10% by weight approximately relative to the total weight of the dye composition, and as a further example, ranging from 0.005% to 5% by weight approximately relative to the total weight of the dye composition.

In general, the acid addition salts which may be used in the context of the dye composition of the invention (oxidation bases and couplers) are chosen, for example, from hydrochlorides, hydrobromides, sulphates, tartrates, lactates, and acetates. The base addition salts which may be used in the context of the dye composition of the invention (oxidation bases and couplers) are, for example, those obtained with sodium hydroxide, potassium hydroxide, aqueous ammonia, and amines.

The medium suitable for dyeing (or support) may comprise water or a mixture of water and at least one organic solvent chosen from (C₁-C₄) lower alkanols, glycerol, glycols, glycol ethers, aromatic alcohols, similar products, and mixtures thereof. The dye composition according to the invention may also contain at least one adjuvant, conventionally used in compositions for dyeing the hair, chosen from anionic surfactants, cationic surfactants, nonionic surfactants, amphoteric surfactants, and zwitterionic surfactants; anionic polymers, cationic polymers, nonionic polymers, amphoteric polymers, zwitterionic polymers; mineral thickeners, organic thickeners; antioxidants; penetrating agents; sequestering agents; fragrances; buffers; dispersants; conditioners chosen from, for example, silicones; film-forming agents; preserving agents; and opacifiers. Needless to say, a person skilled in the art will take care to select this or these optional additional compounds such that the advantageous properties intrinsically associated with the oxidation dye composition in accordance with the invention are not, or are not substantially, adversely affected by the addition(s) envisaged.

The dye composition according to the invention may be in various forms, chosen from liquids, creams, gels, and any other form which is suitable for dyeing keratin fibers, and for example human hair.

A subject of the invention is also a process for dyeing keratin fibers, for example human keratin fibers chosen from hair, using the dye composition as defined above.

According to this process, at least one dye composition as defined above, is applied to the fiber, for a time which is sufficient to develop the desired coloration, either in air or using an oxidizing agent. The dye composition may optionally contain at least one oxidation catalyst, in order to accelerate the oxidation process.

According to a first embodiment of the process of the invention, the coloration of the fiber can be carried out without addition of at least one oxidizing agent, merely by contact with atmospheric oxygen.

According to a second embodiment of the process of the invention, at least one dye composition as defined above is applied to the fiber, the colour being developed at one of acidic, neutral, and alkaline pH using at least one oxidizing agent which is added to the dye composition either just at the time of use, or which is present in at least one oxidizing composition which is applied simultaneously or sequentially in a separate manner.

According to this second embodiment of the dyeing process of the invention, the dye composition described above can be mixed, at the time of use, with at least one oxidizing composition containing, in a medium which is suitable for dyeing, at least one oxidizing agent present in an amount which is sufficient to develop a coloration. The mixture obtained is then applied to the keratin fibers and is, for example, left in place for 3 to 50 minutes approximately, as a further example for 5 to 30 minutes approximately, after which the fiber are rinsed, washed with shampoo, rinsed again, and dried.

The at least one oxidizing agent present in the at least one oxidizing composition as defined above can be chosen from oxidizing agents conventionally used for the oxidation dyeing of keratin fibers, and among which mention may be made of hydrogen peroxide, urea peroxide, alkali metal bromates and persalts chosen from perborates and persulphates. Hydrogen peroxide is particularly preferred.

The pH of the at least one oxidizing composition containing the at least one oxidizing agent as defined above is such that, after mixing with the dye composition, the pH of the resultant composition applied to the keratin fibers ranges, for example, from 3 to 12 approximately, and as a further example, ranges from 5 to 11. It is adjusted to the desired value using at least one of an acidifying agent and a basifying agent commonly used to dye keratin fibers and as defined above.

The at least one oxidizing composition as defined above can also contain at least one adjuvant conventionally used in compositions for dyeing the hair and as defined above.

The composition which is finally applied to the keratin fibers can be in various forms chosen from liquids, creams, gels, and any other form which is suitable for dyeing keratin fibers, for example human hair.

Another subject of the invention is a multi-compartment dyeing device or “kit” or any other multi-compartment packaging system, comprising a first compartment comprising the dye composition as defined above and a second compartment comprising the at least one oxidizing composition as defined above. These devices can be equipped with a means for delivering the desired mixture onto the hair, chosen from the devices described in patent FR 2,586,913, the disclosure of which relating to such devices is hereby incorporated by reference.

The examples which follow are intended to illustrate the invention without, however, limiting its scope.

### EXAMPLES 1 TO 11

### Dyeing in Alkaline Medium

The dye formulations below were prepared:

Common dye support:

| | | |
|---|---|---|
| 5-methylpyrazolo(1,5)-a]pyrimidine- | 0.708 | g |
| 3,7-diamine | | |
| Coupler | y | g |
| Benzyl alcohol | 2 | g |
| Polyethylene glycol 8 EO | 3 | g |
| Ethanol | 18 | g |
| (C₈-C₁₀alkyl polyglucoside as an aqueous | 5 | g A.M. |
| solution containing 60% active material, | | |
| buffered with ammonium citrate, sold under | | |
| the name ORAMIX CG110 by SEPPIC | | |
| Aqueous ammonia containing 20% NH₃ | 10 | g |
| Sodium metabisulphite | 0.205 | g |

| | |
|---|---|
| Sequestering agent | q.s. |

| | | |
|---|---|---|
| Demineralized water | q.s. 100 | g |

DYEING AT ALKALINE pH

| | Coupler combined with | Amount introduced |
|---|---|---|
| | 5-methyl-pyrazolo(1,5- | into the formulation |
| Examples | a)pyrimidine-3,7-diamine | (in g %)-value of y |
| Example 1 | Resorcinol | 0.330 |
| Example 2 | meta-Aminophenol | 0.327 |
| Example 3 | 2-Methyl-5-aminophenol | 0.369 |
| Example 4 | 2-Methylresorcinol | 0.372 |
| Example 5 | 6-Hydroxyindole | 0.399 |
| Example 6 | 2,4-Diaminophenoxyethanol | 0.723 |
| | dihydrochloride | |
| Example 7 | 6-Hydroxybenzomorpholine | 0.453 |
| Example 8 | 2-Amino-3-hydroxypyridine | 0.330 |
| Example 9 | 5-(N-Methyl-3′-pyridyl)methyl-1- | 0.866 |
| | methyl-4-hydroxyindole chloride | |
| Example 10 | 3,6-Dimethyl-1H-pyrazolo(5,1- | 0.408 |
| | c)(1,2,4)-triazole | |
| Example 11 | 6-Chloro-2-methyl-5-aminophenol | 0.582 |

At the time of use, each dye composition 1 to 11 was mixed, weight for weight, with a 20-volume aqueous hydrogen peroxide solution (6% by weight) whose pH was adjusted to about 2.5 with orthophosphoric acid.

The mixture was applied to permanent-waved or natural grey hair containing 90% white hairs, at a rate of 300 mg per 30 mg of hair, for 30 minutes.

The hair was then rinsed, washed with a standard shampoo and dried.

The locks of hair were dyed in the shades given in the table below:

SHADES OBTAINED AT ALKALINE pH

| | |
|---|---|
| Example 1 | Coppery mahogany |
| Example 2 | Coppery mahogany |
| Example 3 | Red-coppery |
| Example 4 | Coppery |
| Example 5 | Golden brown |
| Example 6 | Purple |
| Example 7 | Iridescent red |
| Example 8 | Iridescent purple |
| Example 9 | Violet |
| Example 10 | Golden coppery |
| Example 11 | Coppery red |

The 5-methylpyrazolo(1,5-a)pyrimidine-3,7-diamine thus made it possible to obtain a wide range of colours at alkaline pH.

### EXAMPLES 12 TO 22

### Dyeing in Neutral Medium

The dye formulations below were prepared:

Common dye support:

| | | |
|---|---|---|
| 5-methylpyrazolo(1,5-a)pyrimidine- | 0.708 | g |
| 3,7-diamine | | |
| Coupler | y | g |
| Benzyl alcohol | 2 | g |
| Polyethylene glycol 8 EO | 3 | g |
| Ethanol | 18 | g |
| (C₈-C₁₀)alkyl polyglucoside as an aqueous | 5 | g A.M. |
| solution containing 60% active material, | | |
| buffered with ammonium citrate, sold under | | |
| the name ORAMIX CG110 by SEPPIC | | |
| Phosphate buffer (1.5 M K₂HPO₄/1 M KH₂PO₄) | 10 | g |
| Sodium metabisulphite | 0.205 | g |

| | |
|---|---|
| Sequestering agent | q.s. |

| | | |
|---|---|---|
| Demineralized water | q.s. 100 | g |

DYEING AT pH = 7

| | Coupler combined with | Amount introduced |
|---|---|---|
| | 5-methyl-pyrazolo(1,5- | into the formulation |
| Examples | a)pyrimidine-3,7-diamine | (in g %)-value of y |
| Example 12 | Resorcinol | 0.330 |
| Example 13 | meta-Aminophenol | 0.327 |
| Example 14 | 2-Methyl-5-aminophenol | 0.369 |
| Example 15 | 2-Methylresorcinol | 0.372 |
| Example 16 | 6-Hydroxyindole | 0.399 |
| Example 17 | 2,4-Diaminophenoxyethanol | 0.723 |
| | dihydrochloride | |
| Example 18 | 6-Hydroxybenzomorpholine | 0.453 |
| Example 19 | 2-Amino-3-hydroxypyridine | 0.330 |
| Example 20 | 5-(N-Methyl-3′-pyridyl)methyl-1- | 0.866 |
| | methyl-4-hydroxyindole chloride | |
| Example 21 | 3,6-Dimethyl-1H-pyrazolo(5,1- | 0.408 |
| | c)(1,2,4)-triazole | |
| Example 22 | 6-Chloro-2-methyl-5-aminophenol | 0.582 |

At the time of use, each dye composition 12 to 22 was mixed, weight for weight, with a 20-volume aqueous hydrogen peroxide solution (6% by weight) whose pH was adjusted to about 2.5 with orthophosphoric acid.

The mixture was applied to permanent-waved or natural grey hair containing 90% white hairs, at a rate of 300 mg per 30 mg of hair, for 30 minutes.

The hair was then rinsed, washed with a standard shampoo and dried.

The locks of hair were dyed in the shades given in the table below:

SHADES OBTAINED AT pH 7

| | |
|---|---|
| Example 12 | Coppery mahogany |
| Example 13 | Coppery mahogany |
| Example 14 | Red-coppery |
| Example 15 | Iridescent coppery |
| Example 16 | Chestnut |
| Example 17 | Purple |
| Example 18 | Iridescent red |
| Example 19 | Iridescent ash |
| Example 20 | Violet |
| Example 21 | Golden coppery |
| Example 22 | Coppery red |

The 5-methylpyrazolo(1,5-a)pyrimidine-3,7-diamine thus made it possible to obtain a wide range of colours at neutral pH.

What is claimed is:

1. Composition for the oxidation dyeing of keratin fibers comprising at least one oxidation base chosen from 5-methylpyrazolo(1,5-a)pyrimidine-3,7-diamine of formula (I): 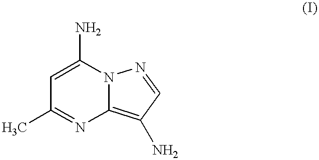
acid addition salts thereof, and base addition salts thereof; the composition containing no coupler chosen from the N-(2-hydroxybenzene)carbamate type and the N-(2-hydroxybenzene)urea type.

2. Composition according to claim 1, further comprising a medium suitable for dyeing.

3. Composition according to claim 1, wherein the at least one oxidation base is present in the composition in an amount ranging from 0.001% to 10% by weight relative to the total weight of the composition.

4. Composition according to claim 3, wherein the at least one oxidation base is present in the composition in an amount ranging from 0.05% to 6% by weight relative to the total weight of the composition.

5. Composition according to claim 2, wherein the medium suitable for dyeing is chosen from water and a mixture of water and at least one organic solvent chosen from (C₁-C₄) lower alkanols, glycerol, glycols, glycol ethers, aromatic alcohols, similar products, and mixtures thereof.

6. Composition according to claim 1, wherein the composition has a pH ranging from 3 to 12.

7. Composition according to claim 1, wherein the composition comprises at least one additional oxidation base chosen from para-phenylenediamines, bis(phenyl)alkylenediamines, para-aminophenols, ortho-aminophenols, and heterocyclic bases other than 5-methylpyrazolo(1,5-a)pyrimidine-3,7-diamine.

8. Composition according to claim 7, wherein the at least one additional oxidation base is present in the composition in an amount ranging from 0.0005% to 12% by weight relative to the total weight of the composition.

9. Composition according to claim 1, wherein the composition comprises at least one coupler and/or at least one direct dye.

10. Composition according to claim 9, wherein the at least one coupler is chosen from meta-phenylenediamines, meta-aminophenols, meta-diphenols, mononaphthalene derivatives, polyhydroxylated naphthalene derivatives, heterocyclic couplers, and acid addition salts thereof.

11. Composition according to claim 9, wherein the at least one coupler is present in the composition in an amount ranging from 0.0001% to 10% by weight relative to the total weight of the composition.

12. Composition according to claim 1, wherein the acid addition salts are chosen from hydrochlorides, hydrobromides, sulphates, tartrates, lactates, and acetates, and wherein the base addition salts are chosen from those obtained with sodium hydroxide, potassium hydroxide, aqueous ammonia, and amines.

13. Process for dyeing keratin fibers, comprising, applying to the fibers a composition for the oxidation dyeing of keratin fibers comprising at least one oxidation base chosen from 5-methylpyrazolo(1,5-a)pyrimidine-3,7-diamine of formula (I): 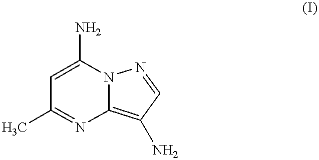
acid addition salts thereof, and base addition salts thereof; the composition containing no coupler chosen from the N-(2-hydroxybenzene)carbamate type and the N-(2-hydroxybenzene)urea type, for a time which is sufficient to develop the desired coloration, optionally in the presence of at least one oxidation catalyst.

14. Process according to claim 13, wherein the keratin fibers are human keratin fibers.

15. Process according to claim 14, wherein the human keratin fibers are hair.

16. Process according to claim 13, wherein the desired coloration is developed merely by contact with atmospheric oxygen.

17. Process according to claim 13, wherein the desired coloration is developed using at least one oxidizing agent which is added to the composition at the time of use.

18. Process according to claim 17, wherein the at least one oxidizing agent, which is present in at least one oxidizing composition, is applied simultaneously or sequentially in a separate manner with respect to the composition for the oxidation dyeing of keratin fibers.

19. Process according to claim 13, wherein the desired coloration is developed with at least one oxidizing agent chosen from hydrogen peroxide, urea peroxide, alkali metal bromates, and persalts.

20. Process according to claim 19, wherein the persalts are chosen from perborates and persulphates.

21. Multi-compartment device or multi-compartment dyeing kit comprising a first compartment comprising a composition for the oxidation dyeing of keratin fibers comprising at least one oxidation base chosen from 5-methylpyrazolo(1,5-a)pyrimidine-3,7-diamine of formula (I): 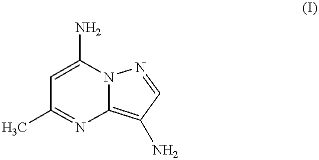
acid addition salts thereof, and base addition salts thereof; the composition containing no coupler chosen from the N-(2-hydroxybenzene)carbamate type and the N-(2-hydroxybenzene)urea type, and a second compartment comprising at least one oxidizing composition.
